(19) United States
(12) Patent Application Publication
(10) Pub. No.: US 2023/0194495 A1
(43) Pub. Date: Jun. 22, 2023

(54) Method for Quantitatively Detecting Acetaldehyde in Wine Samples Using Fluorescent Probes

(71) Applicant: Jiangnan University, Wuxi (CN)

(72) Inventors: Chunfeng LIU, Wuxi (CN); Yisong LIU, Wuxi (CN); Shanshan CHEN, Wuxi (CN); Qi LI, Wuxi (CN); Jinjing WANG, Wuxi (CN); Feiyun ZHENG, Wuxi (CN); Chengtuo NIU, Wuxi (CN); Xin Xu, Wuxi (CN)

(21) Appl. No.: 18/155,227

(22) Filed: Jan. 17, 2023

Related U.S. Application Data
(63) Continuation of application No. PCT/CN2022/081626, filed on Mar. 18, 2022.

(30) Foreign Application Priority Data
Dec. 1, 2021 (CN) 2021114537799

(51) Int. Cl. G01N 33/14; G01N 21/64

(52) U.S. Cl. CPC G01N 33/146; G01N 21/643

(57) ABSTRACT

Disclosed in the disclosure is a method for detecting acetaldehyde in wine samples using fluorescent probes, belonging to the field of wine quality control. The detection method of the disclosure is used for measuring acetaldehyde in wines based on fluorescent probes. Under the acidic condition of pH=2.0, the specific binding between the fluorescent probe and acetaldehyde is realized according to the principle of photoelectron induced transfer. The fluorescent probe has a good linear relationship with the concentration of acetaldehyde in a range of 0-200 mg/L, the limit of detection (LOD) is 3.6×10⁻⁸ mol/L, and the recovery rate of samples is 94.02-108.12%. The detection method has the advantages including low cost, wide linear range, high sensitivity, and being fast and accurate. The fluorescent probe is successfully applied to the detection and analysis of wine samples and samples in a beer fermentation process.

### TECHNICAL FIELD

The disclosure relates to a method for quantitatively detecting acetaldehyde in wine samples using fluorescent probes, belonging to the field of wine quality control.

### BACKGROUND

Flavor substances of wines play an extremely important role in taste and aroma. Aldehydes are main carbonyl compounds existing in wines, wherein acetaldehyde is a volatile aldehyde with the highest content in wines, and accounts for 60-90% of the total content of aldehydes in beer, Baijiu (Chinese liquor), Huangjiu (Chinese rice wine) and grape wine.

Acetaldehyde was recognized as a Class 2B carcinogen by the International Agency for Research on Cancer (IARC), that is, acetaldehyde may cause cancers in humans. In 2009, acetaldehyde was listed as a Class I carcinogen, which means that there was sufficient evidence for human carcinogenesis. The content of acetaldehyde directly affects the flavor and aging of wines. When the concentration of acetaldehyde is low, there is a fruit fragrance. When the concentration of acetaldehyde is high, it will produce a pungent and irritating smell and bring a bad grass smell to wines, shorten the fresh-keeping period of the wine flavor, and even cause adverse reactions to the human body after drinking.

So far, there are many methods for detecting the content of acetaldehyde in wine samples, and the most common methods are gas chromatography (GC) and high performance liquid chromatography (HPLC). In order to improve the accuracy, the GC and HPLC are usually combined with a solid phase microextraction or derivatization treatment technology. However, the GC and HPLC involve the use of expensive instruments, complex operating procedures and longer detection time. Furthermore, in order to ensure the accuracy of detection, it is usually necessary to debug and maintain devices, resulting in higher detection cost. Therefore, it is imperative to establish a method for efficient analysis and low-cost detection of acetaldehyde in wine samples, which is helpful to effectively control the content of acetaldehyde in wines, thereby improving the flavor of wines.

### SUMMARY

The technical problem to be solved by the disclosure is to provide a method for detecting acetaldehyde in wine samples using fluorescent probes. The fluorescent probe has weaker fluorescence, the fluorescence is enhanced after interacting with acetaldehyde, and the fluorescence intensity is directly proportional to the concentration of acetaldehyde to realize the quantification of the concentration of acetaldehyde, so that the evaluation of the content of acetaldehyde in wine products is more efficient, scientific and comprehensive.

The fluorescent probes involved in the disclosure have the following structural formula:

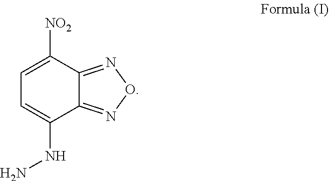 [SMILES: NNc1ccc([N+](=O)[O-])c2nonc12]

A method for detecting the content of acetaldehyde in wine samples using fluorescent probes, provided by the disclosure, comprises the following steps:

(1) dispersing fluorescent probes with the structure shown in Formula (I) in organic solvents to obtain a fluorescent probe solution, then, mixing the fluorescent probe solution, a hydrochloric acid solution and a series of acetaldehyde standard solutions with known concentrations respectively for reaction at 0-10° C., and obtaining a mixed system after the reaction;

(2) measuring the fluorescence intensity of the mixed system on a fluorescence spectrometer, and linearly correlating the fluorescence intensity with the concentration of the corresponding acetaldehyde standard solution to obtain a quantitative detection model; and

(3) mixing the fluorescent probe solution, the hydrochloric acid solution and the wine samples for reaction according to the process in step (1), then, measuring the fluorescence intensity of the mixed system, and calculating the concentration of acetaldehyde in the wine samples by the quantitative detection model in step (2).

In an embodiment of the disclosure, the volume ratio of the fluorescent probe solution to the hydrochloric acid solution to the acetaldehyde standard solution is 2:1:1.

In an embodiment of the disclosure, the organic solvents in step (1) are acetonitrile and dimethyl sulfoxide (DMSO), and the volume ratio of the acetonitrile to the DMSO is 10:1.

In an embodiment of the disclosure, the concentration of the fluorescent probe solution is 600 mg/L.

In an embodiment of the disclosure, the hydrochloric acid solution is prepared by taking acetonitrile as a solvent.

In an embodiment of the disclosure, the pH of the hydrochloric acid solution is 2, and the mass fraction is 2.96%.

In an embodiment of the disclosure, the concentration range of a series of acetaldehyde standard solutions with known concentrations is 0-200 mg/L, specifically may be 0 mg/L, 10 mg/L, 20 mg/L, 50 mg/L, 100 mg/L, 150 mg/L, and 200 mg/L.

In an embodiment of the disclosure, the reaction time is 40-60 min, specifically may be 50 min.

In an embodiment of the disclosure, the reaction temperature specifically may be 5° C.

In an embodiment of the disclosure, the fluorescence intensity is the fluorescence intensity at the emission wavelength of 553 nm.

In an embodiment of the disclosure, the quantitative detection model is F_{553 nm}=346.14C+45.17, R²=0.9954, and the unit of C is mg/L.

In an embodiment of the disclosure, the excitation wavelength is 485 nm, and the emission wavelength is 553 nm.

In an embodiment of the disclosure, the wine samples need to be pretreated as follows:

adding a drop of defoamer to aerated beer samples, and diluting Baijiu, Huangjiu and grape wine samples 25 times with distilled water; taking 50 mL of wine sample, and distilling the wine sample with a diacetyl distilling apparatus; and stopping receiving when the content of a distillate is close to 10 mL, and complementing to 10 mL with distilled water to obtain a distillate A, that is, a wine sample.

In an embodiment of the disclosure, the distillation process is completed within 1 min, and the distillate is received in an ice bath by a 10 mL colorimetric tube with a stopper.

In an embodiment of the disclosure, the wine samples comprise Baijiu, Huangjiu and grape wine samples.

In an embodiment of the disclosure, the detection method comprises the following specific processes:

(1) preparing a hydrochloric acid solution with a mass fraction of 2.96% by taking acetonitrile as a solvent; preparing a fluorescent probe solution with a concentration of 600 mg/L by taking acetonitrile as a solvent and DMSO as a cosolvent (10:1, v/v);

(2) preparing acetaldehyde solutions with concentrations of 0 mg/L, 10 mg/L, 20 mg/L, 50 mg/L, 100 mg/L, 150 mg/L, and 200 mg/L by using distilled water; adding 50 μL of hydrochloric acid solution, 50 μL of standard acetaldehyde solution, and 100 μL of fluorescent probe solution to a 96-well enzyme linked immunosorbent assay (ELISA) plate, and performing a low temperature reaction in an incubator at 5° C. for 50 min; measuring fluorescence intensity F_{553 nm} on a fluorescence spectrometer, and obtaining a standard working curve by taking the concentration C of acetaldehyde as a horizontal coordinate and the fluorescence intensity as a vertical coordinate, wherein a linear regression equation is:

F_{553 nm}=346.14C+45.17, R²=0.9954, and the unit of C is mg/L;

(3) taking 50 mL of wine sample (adding a drop of defoamer to aerated beer samples, and diluting Baijiu, Huangjiu and grape wine samples 25 times with distilled water), and distilling the wine sample with a diacetyl distilling apparatus; stopping receiving when the content of a distillate is close to 10 mL, and complementing to 10 mL with distilled water to obtain a distillate A; and

(4) adding 50 μL of hydrochloric acid solution, 50 μL of distillate A, and 100 μL of fluorescent probe solution to the 96-well ELISA plate, performing a low temperature reaction in the incubator at 5° C. for 50 min, then performing detection on the fluorescence spectrometer, and substituting the fluorescence intensity into the linear regression equation in step (2) to obtain the concentration C, wherein the concentration of the sample is:

Cₛₐₘₚₗₑ=C/N (N represents the concentration multiple of the sample, when the sample is beer, N=5, and when the sample is Baijiu, Huangjiu or grape wine, N=0.2).

In an embodiment of the disclosure, the fluorescent probe may be purchased or self-made. A self-made synthesis method comprises: weighing and putting 500 mg of NBD-Cl in a flask, and wrapping an outer wall with tin foil to avoid light; adding 50 ml of chloroform to fully dissolve NBD-Cl; adding 50 ml of 5% hydrazine hydrate (3.1 ml of 80% hydrazine hydrate and 46.9 ml of methanol); after nitrogen purging, sealing the flask, allowing the flask to stand for 1 h to precipitate yellowish-brown sediments, and performing suction filtration; and cleaning the filter cake with dichloromethane to obtain a solid probe product. The corresponding synthesis route is:

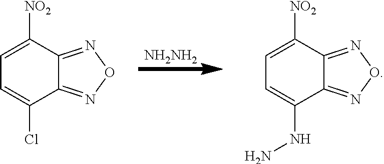

The principle of detecting acetaldehyde using fluorescent probes in the disclosure is as follows: a strong electron donating group (hydrazine group) provides electrons to an electron withdrawing group (nitro group), a fluorescence group is affected by a photoelectron induced transfer effect, the radiative transition of the electrons is blocked, and the fluorescence is inhibited. After the fluorescent probe interacts with acetaldehyde, the hydrazine group no longer provides electrons to the nitro group so as to cut off the photoelectron induced transfer effect and restore the fluorescence, so that the fluorescent probe belongs to an enhanced fluorescent probe. The disclosure uses fluorescent probes to detect acetaldehyde in wine samples. Benzoxadiazole belongs to a relatively common fluorescence group, and changing 4-bit and 7-bit substituent groups will produce different emission characteristics. In the disclosure, a 4-bit substituent group is designed as a nitro group as a strong electron withdrawing group, and a 7-bit substituent group is designed as a hydrazine group as a reactive group to form a photoinduced electron transfer effect. After the reactive group is combined with acetaldehyde, acetaldehyde competes for the electrons transferred from the hydrazine group to the nitro group, and then, the photoinduced electron transfer process is destroyed, resulting in enhanced fluorescence emission. By means of this principle, the detection and analysis of the content of acetaldehyde in wine samples can be realized.

The disclosure has the following beneficial effects:

1. The synthesis route of the fluorescent probe provided is simple, and the method provided is cheap, simple and efficient.

2. The method provided can solve the problem of background interference, and the method of removing background interference by means of distillation is proposed for the first time in the application of fluorescent probes to detect actual samples.

3. The method provided has higher effectiveness: the limit of detection (LOD) of acetaldehyde is 3.6×10⁻⁸ mol/L; the fluorescent probe has a good linear relationship with the concentration of acetaldehyde in a range of 0-200 mg/L, and the linear range is wide; precision experiment results show that the RSD is 5.30% in a simulated solution system, and the RSD is 3.72% in a real wine sample system; and the recovery rate is 93.87-99.75% in the simulated solution system, and by contrast, the recovery rate is 94.02-108.12% in the real wine sample system. The evaluation method provided by the disclosure is applied to the detection and analysis of finished wine samples and samples in a beer fermentation process.

### BRIEF DESCRIPTION OF FIGURES

FIG. 1 shows a fluorescence selectivity diagram of fluorescent probes, wherein the excitation wavelength is 485 nm, and the emission wavelength is 553 nm.

FIG. 2 shows an interference resistance diagram of fluorescent probes, wherein the excitation wavelength is 485 nm, and the emission wavelength is 553 nm.

FIG. 3 shows standard curve and linear range diagrams of fluorescent probes for recognizing acetaldehyde, wherein the excitation wavelength is 485 nm, and the emission wavelength is 553 nm.

FIG. 4 shows a relationship between the concentration multiple and the recovery rate in a distillation process, wherein the excitation wavelength is 485 nm, and the emission wavelength is 553 nm.

FIG. 5 shows diagrams of applicable ranges of detection conditions of fluorescent probes for recognizing acetaldehyde, wherein the excitation wavelength is 485 nm, and the emission wavelength is 553 nm.

FIG. 6 shows the reproducibility of a method for recognizing acetaldehyde using fluorescent probes, wherein the excitation wavelength is 485 nm, and the emission wavelength is 553 nm.

FIG. 7 shows a tracking detection diagram of acetaldehyde in a beer fermentation process using fluorescent probes, wherein the excitation wavelength is 485 nm, and the emission wavelength is 553 nm.

### DETAILED DESCRIPTION

### Example 1: Construction of Quantitative Detection Model

(1) A hydrochloric acid solution (pH=2) with a mass fraction of 2.96% was prepared by taking acetonitrile as a solvent; a fluorescent probe solution with a concentration of 600 mg/L was prepared by taking acetonitrile as a solvent and DMSO as a cosolvent (10:1, v/v);

(2) a series of acetaldehyde standard solutions with concentrations of 0 mg/L, 10 mg/L, 20 mg/L, 50 mg/L, 100 mg/L, 150 mg/L, and 200 mg/L were prepared by using distilled water; 50 μL of hydrochloric acid solution (the concentration of hydrochloric acid was 2.96 wt %), 50 μL of acetaldehyde standard solution and 100 μL of fluorescent probe solution were added to a 96-well ELISA plate, and a low temperature reaction was performed in an incubator at 5° C. for 50 min to obtain a mixed system;

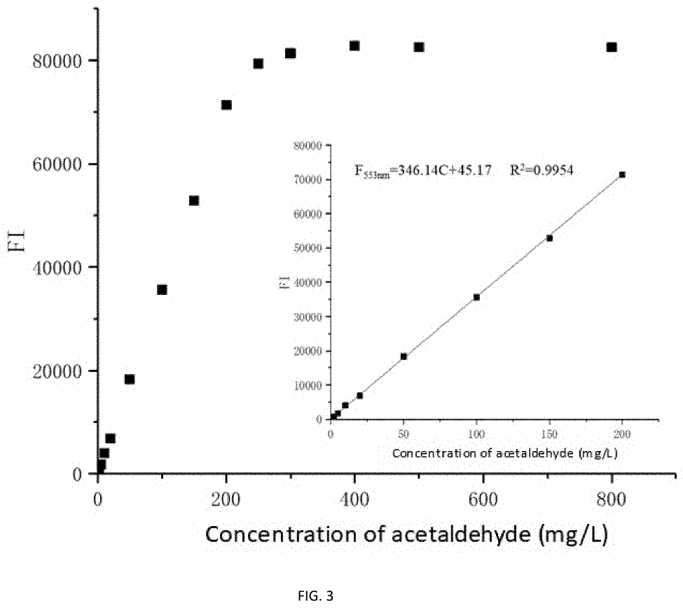

(3) the fluorescence intensity F_{553 nm} of the mixed system at 553 nm was measured on a fluorescence spectrometer, and a standard working curve (as shown in FIG. 3) was obtained by taking the concentration C of acetaldehyde as a horizontal coordinate and the fluorescence intensity as a vertical coordinate, wherein a linear regression equation was: F_{553 nm}=346.14C+45.17, R²=0.9954, and the unit of C was mg/L. The linear range of acetaldehyde detection was 0-200 mg/L, the linear range was wide, and the LOD was 3.6×10⁻⁸ mol/L.

### Example 2: Detection of Acetaldehyde in Simulated Wine Samples

The content of acetaldehyde in samples was measured by fluorescent probes. Specific operation processes and experiment conditions were as follows:

### 1. Sample Treatment:

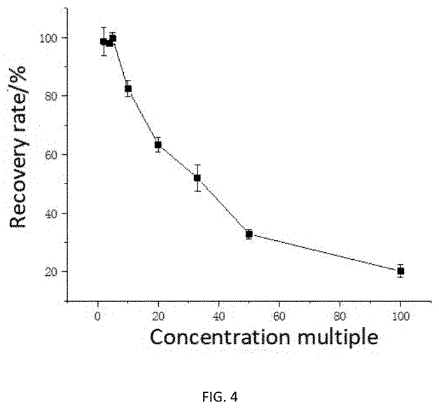

500 μL of acetaldehyde solution (1000 mg/L) was added to 50 ml of distilled water to prepare a standard solution with a concentration of 10 mg/L, and a standard sample was distilled with a diacetyl distilling apparatus. The results of the concentration multiple and recovery rate are shown in FIG. 4, and the results show that the recovery rate is better when the concentration multiple is 5 or less. In consideration of the distillation efficiency, the concentration multiple should be 5.

Specific operations were as follows: 50 mL of wine sample was taken (a drop of defoamer needs to be added for aerated beer samples, and Baijiu, Huangjiu and grape wine samples need to be diluted 25 times with distilled water), and the wine sample was distilled with a diacetyl distilling apparatus; and the distillate was not received when the content of the distillate was close to 10 mL, and was complemented to 10 mL with distilled water to obtain a distillate A for later use.

### 2. Detection Conditions of 96-Well ELISA Plate:

50 μL of acid solution, 50 μL of distillate A and 100 μL of fluorescent probe solution were put in an incubator at 5° C. for a low temperature reaction for 50 min. The fluorescence intensity was measured on a fluorescence spectrometer, wherein the excitation wavelength was 485 nm, and the emission wavelength was 553 nm.

### 3. Calculation of Content of Acetaldehyde:

The fluorescence intensity was substituted into the linear regression equation F_{553 nm}=346.14C+45.17 to obtain the concentration C, wherein the unit of C was mg/L, and the concentration of the sample was:

Cₛₐₘₚₗₑ=C/N (N represents the concentration multiple of distillation, when the sample was beer, N=5, and when the sample was Baijiu, Huangjiu or grape wine, N=0.2).

### 4. Optimization of Detection Conditions:

In order to avoid the interference caused by the difference between different samples, this part of the experiment uses the same beer sample to optimize the detection conditions. Optimization factors include temperature, reaction time, acid concentration and probe concentration.

### (1) Temperature

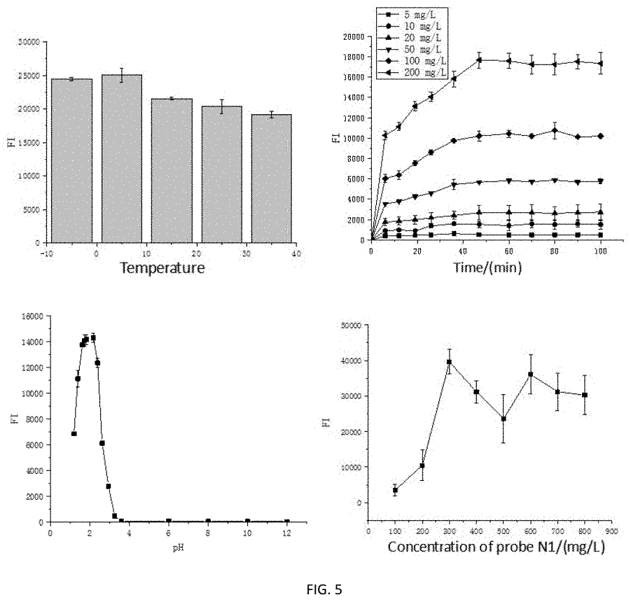

This experiment was designed to react at 0° C., 5° C., 15° C., 25° C. and 35° C., and then, the response value of acetaldehyde in the sample was measured, as shown in FIG. 5. The research shows that with the gradual increase of the system temperature, the response value of acetaldehyde decreases gradually, and the response value of acetaldehyde changes little at 0° C. and 5° C. From the perspective of energy consumption, when the balance temperature was 5° C., the detection effect was the best, and the sensitivity was the highest.

### (2) Reaction Time

The change of the response value of acetaldehyde in the sample was investigated at different balance times of 0 min, 5 min, 10 min, 20 min, 25 min, 40 min, 50 min, 60 min, 70 min, 80 min, 90 min and 100 min, as shown in FIG. 5. The results show that when the balance time was 0-50 min, the response value of acetaldehyde increases with the increase of the time; and when the balance time was greater than 50 min, the response value of acetaldehyde changes little, and the probe reacts completely with acetaldehyde, so the preferred reaction time was 50 min.

(3) pH

The probe has certain pH sensitivity. The response of the probe to acetaldehyde (10 mg/L) was evaluated at different pH, as shown in FIG. 5. The results show that when pH=2.0, the response value of acetaldehyde reaches the maximum. Therefore, in this research, the detection was performed under the condition that pH=2.0, and the mass fraction of the hydrochloric acid solution was 2.96%.

### (4) Probe Concentration

The probe solution was yellowish, and the background interference of the system will affect the final fluorescence intensity. This experiment researches the change of the response value of acetaldehyde (100 mg/L) in an acetonitrile solution with pH=2.0 at the probe concentrations of 100 mg/L, 200 mg/L, 300 mg/L, 400 mg/L, 500 mg/L, 600 mg/L, 700 mg/L and 800 mg/L, as shown in FIG. 5. The results show that when the probe concentration was 300 mg/L, the response value of acetaldehyde was the largest, and when the probe concentration was 600 mg/L, the response value was the second largest. The further research shows that the probe concentration of 300 mg/L only presents a good linear relationship within 0-80 mg/L, F_{553 nm}=305.03C+80.15, and R²=0.9923; and the probe concentration of 600 mg/L may present a good linear relationship within 0-200 mg/L, F_{553 nm}=346.14C+45.17, and R²=0.9954. From the perspective of the linear range, the probe concentration of 600 mg/L further conforms to actual requirements, especially for wine samples with higher content of acetaldehyde such as Baijiu.

### Example 3: Standard Recovery Verification of Detection Method

The recovery rate of the method was investigated in a simulated solution system and a real wine sample system respectively. In an acetaldehyde simulated system: 50 μL/200 μL of acetaldehyde stock solution (1 g/L) was added to 50 mL of acetaldehyde standard solution sample (10 mg/L) respectively, and 3 parallel samples were prepared for each standard volume. In a real wine system, 50 μL/100 μL/200 μL of acetaldehyde stock solution (1 g/L) was added to 50 mL of beer, 500 μL/1000 μL/2000 μL of acetaldehyde stock solution (1 g/L) was added to 50 mL of Baijiu, 250 μL/500 μL/1000 μL of acetaldehyde stock solution (1 g/L) was added to 50 mL of Huangjiu and grape wine, and 3 parallel samples were prepared for each standard volume. Then, the content of acetaldehyde was detected by the above detection method using fluorescent probes. The results are shown in Table 1.

TABLE 1
Experimental results of standard recovery rate (n = 3)

| | | Standard | Detection | Recovery |
|---|---|---|---|---|
| | Sample | volume | value | rate |
| No. | (mg/L) | (mg/L) | (mg/L) | (%) |
| 1 | 10.00 | 1 | 10.95 ± 0.42 | 95.36 |
| 2 | 10.00 | 2 | 11.99 ± 0.24 | 99.75 |
| 3 | 10.00 | 4 | 13.76 ± 0.36 | 93.87 |
| 4 | 9.52 | 1 | 10.48 ± 0.15 | 96.22 |
| 5 | 9.52 | 2 | 11.48 ± 0.21 | 98.31 |
| 6 | 9.52 | 4 | 13.42 ± 0.09 | 97.48 |
| 7 | 122.09 | 10 | 129.49 ± 1.88 | 98.03 |
| 8 | 122.09 | 20 | 153.63 ± 2.04 | 108.12 |
| 9 | 122.09 | 40 | 163.81 ± 2.12 | 101.06 |
| 10 | 49.17 | 5 | 51.11 ± 1.65 | 94.36 |
| 11 | 49.17 | 10 | 55.90 ± 1.22 | 94.47 |
| 12 | 49.17 | 20 | 66.49 ± 1.41 | 96.12 |
| 13 | 32.44 | 5 | 38.81 ± 0.66 | 103.65 |
| 14 | 32.44 | 10 | 41.82 ± 1.53 | 98.55 |
| 15 | 32.44 | 20 | 49.30 ± 0.92 | 94.02 |

Note:
samples 1-3 are acetaldehyde standard solutions, samples 4-6 are beer samples, samples 7-9 are Baijiu samples, samples 10-12 are Huangjiu samples, and samples 13-15 are grape wine samples.

It can be seen from Table 1 that the recovery rates of acetaldehyde in the simulated solution system and the real wine sample system are 93.87-99.75% and 94.02-108.12% respectively. The method is higher in recovery rate and better in effectiveness.

### Example 4: Selectivity of Detection Method to Different Interference Analytes

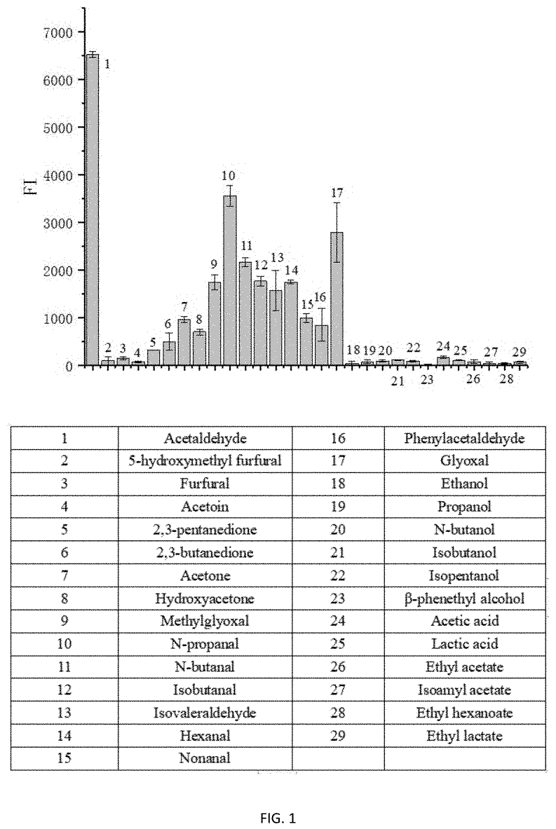

50 μL of hydrochloric acid solution H and 100 μL of fluorescent probe solution were added to a 96-well ELISA plate, and 50 μL of the following analytes with the same mass concentration (15 mg/L) were added: acetaldehyde, 5-hydroxymethyl furfural, furfural, acetoin, 2,3-pentanedione, 2,3-butanedione, acetone, hydroxyacetone, methylglyoxal, n-propanal, n-butanal, isobutanal, isovaleraldehyde, hexanal, nonanal, phenylacetaldehyde, glyoxal, propanol, n-butanol, isobutanol, isopentanol, β-phenethyl alcohol, acetic acid, lactic acid, ethyl acetate, isoamyl acetate, ethyl hexanoate, and ethyl lactate. A low temperature reaction was performed in an incubator at 5° C. for 50 min, and the fluorescence intensity was measured on a fluorescence spectrometer. It can be seen from FIG. 1 that under the same mass concentration, the probe has the highest response value to acetaldehyde, and has a higher response value to some microgram carbonyl compounds in beer, such as n-propanal, n-butanal and isobutanal, but the response value is lower than that of acetaldehyde; the response value of the probe to milligram carbonyl compounds in beer is lower, which is only 3.5-7.6% of the response value of acetaldehyde; and furthermore, it can be found that the probe has almost no response to alcohol and ester substances, and has very low response value to main flavor substances except acetaldehyde in Baijiu, Huangjiu and grape wine.

### Example 5: Detection of Acetaldehyde in Real Wine Samples

23 kinds of wine samples were collected, and the content of acetaldehyde in the wine samples was detected according to the sample detection method recorded in steps (1), (2) and (3) in Example 4. The results are shown in Table 2, and the average values and standard deviations of various samples are shown in Table 3.

TABLE 2
Content of acetaldehyde in different wine samples (n = 3)

| | Detection results using | Detection results using |
|---|---|---|
| | fluorescent probes | gas chromatography |
| No. | (mg/L) | (mg/L) |
| 1 | 10.23 ± 0.76 | 15.00 ± 0.63 |
| 2 | 8.82 ± 0.59 | 10.46 ± 0.82 |
| 3 | 19.73 ± 2.13 | 17.94 ± 1.34 |
| 4 | 18.82 ± 1.48 | 25.24 ± 1.86 |
| 5 | 12.25 ± 0.72 | 9.50 ± 0.31 |
| 6 | 28.61 ± 0.65 | 41.23 ± 3.2 |
| 7 | 12.61 ± 1.42 | 15.21 ± 0.88 |
| 8 | 16.69 ± 0.04 | 24.10 ± 0.92 |
| 9 | 26.20 ± 0.22 | 28.35 ± 0.82 |
| 10 | 12.05 ± 0.41 | 15.03 ± 0.35 |
| 11 | 13.5 ± 0.45 | 17.20 ± 0.43 |
| 12 | 11.7 ± 0.22 | 14.57 ± 0.68 |
| 13 | 16.55 ± 0.42 | 23.95 ± 0.22 |
| 14 | 18.55 ± 0.76 | 23.94 ± 0.31 |
| 15 | 19.45 ± 0.45 | 17.21 ± 0.66 |
| 16 | 21.62 ± 1.12 | 25.25 ± 0.56 |
| 17 | 24.17 ± 0.88 | 31.51 ± 1.10 |
| 18 | 181.08 ± 3.67 | 194.66 ± 2.98 |
| 19 | 122.09 ± 3.12 | 147.33 ± 3.01 |
| 20 | 81.00 ± 2.91 | 93.30 ± 1.26 |
| 21 | 49.17 ± 2.35 | 53.79 ± 1.66 |
| 22 | 21.13 ± 1.17 | 30.27 ± 2.10 |
| 23 | 32.44 ± 3.24 | 31.65 ± 2.64 |

Note:
samples 1-17 are beer samples, samples 18-19 are Baijiu samples, samples 20-21 are Huangjiu samples, and samples 22-23 are grape wine samples.

TABLE 3
Average values and standard deviations of content
of acetaldehyde in different wine samples

| Samples | Average value (mg/L) | RSD(%) |
|---|---|---|
| Beer samples | 17.80 | 4.05 |
| Baijiu samples | 151.59 | 2.03 |
| Huangjiu samples | 65.09 | 3.59 |
| Grape wine samples | 26.79 | 5.22 |

It can be seen from Table 2 that acetaldehyde can be detected in 23 kinds of wine samples, and in the beer, Baijiu, Huangjiu and grape wine samples, the average content of acetaldehyde is 17.80 mg/L, 151.59 mg/L, 65.09 mg/L and 26.79 mg/L respectively, and the average RSD is 3.72%. Through significant difference analysis of SPSS, Sig.=0.756>0.05, and Sig.(double tail)=0.666>0.05, indicating that there is no significant difference between the detection results of the two methods. Acetaldehyde is mainly produced by biological and chemical ways in the process of wine brewing. Real-time monitoring of the content of acetaldehyde is of great significance for wine quality control. When the fluorescent probe method is used for detecting the content of acetaldehyde in wine products, the accuracy of the measured result can be ensured, the use of expensive large-scale detection instruments is avoided, the method is simple and fast, and the cost is saved.

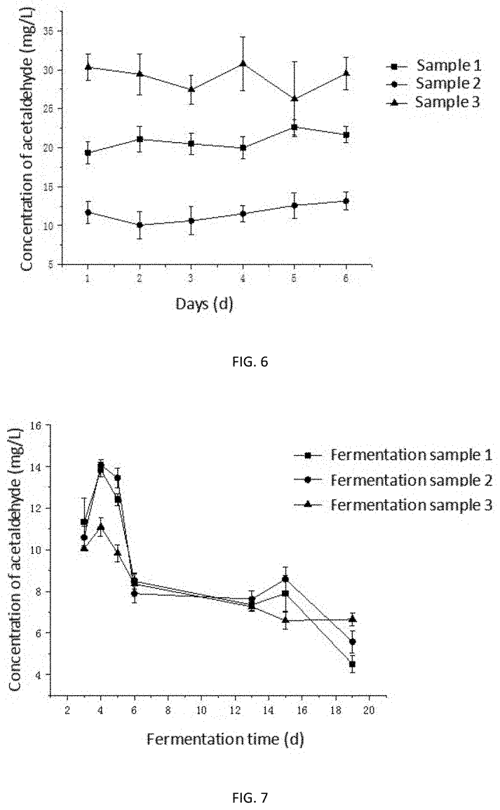

Three samples were selected to test the reproducibility of the detection method. The three samples were frozen, and 6 times of parallel detection were performed within 6 days. The results are shown in FIG. 6, indicating that the reproducibility of the detection method is better.

### Example 6: Detection of Acetaldehyde in Beer Fermentation Process

Three kinds of different beer yeasts were inoculated into 12° P wort for a fermentation experiment, wherein the inoculation volume was 1.5×10⁷ CFU/mL. During the fermentation process, according to the sample detection method recorded in steps (1), (2) and (3) in Example 4, sampling analysis was performed every day to obtain the content of acetaldehyde in fermentation liquor. The results are shown in FIG. 6.

It can be seen from FIG. 7 that during the fermentation process, the content of acetaldehyde first increases to the maximum value, and then slowly decreases. The concentration of acetaldehyde of the three kinds of fermentation liquor samples reaches a peak value after 4 days of fermentation.

### Example 7: Interference Resistance of Detection Method to Carbonyl Compounds and Main Flavor Substances in Wine Samples

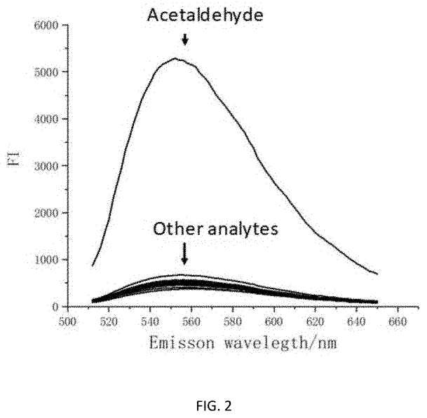

Real wine samples were simulated to verify the interference resistance of the probe. Interference substances shall include carbonyl compounds and alcohol and ester compounds rich in wine samples, the concentration was selected according to the highest reported content, and the content of acetaldehyde was based on the average concentration of each kind of wines. Taking a beer anti-interference analysis sample as an example, the concentration of each analyte was as follows: the concentration of acetaldehyde was 10 mg/L, the concentration of propanal was 0.3 mg/L, the concentration of n-butanal was 0.3 mg/L, the concentration of isobutanal was 0.3 mg/L, the concentration of isovaleraldehyde was 0.1 mg/L, the concentration of heptaldehyde was 0.2 mg/L, the concentration of octanal was 0.2 mg/L, the concentration of furfural was 2 mg/L, the concentration of 5-hydroxymethyl furfural was 8 mg/L, the concentration of 2,3-butanedione was 1 mg/L, the concentration of 2,3-pentanedione was 0.5 mg/L, the concentration of acetoin was 5 mg/L, the concentration of methylglyoxal was 0.1 mg/L, the concentration of n-propanol was 25 mg/L, the concentration of isopentanol was 100 mg/L, the concentration of ethyl acetate was 50 mg/L, and the concentration of isoamyl acetate was 10 mg/L. 50 μL of acid solution H and 100 μL of fluorescent probe solution were added to a 96-well ELISA plate, the above anti-interference analytes were added respectively, a low temperature reaction was performed in an incubator at 5° C. for 50 min, and a fluorescence spectrogram was measured on a fluorescence spectrometer. It can be found from FIG. 2 that only acetaldehyde shows a relatively strong fluorescence signal at 553 nm, so the fluorescent probe is suitable for complex systems of wine samples such as beer samples.

Although the disclosure has been disclosed above with preferred examples, it is not intended to limit the disclosure. Anyone familiar with this technology can make various changes and modifications without departing from the spirit and scope of the disclosure, therefore, the protection scope of the disclosure should be defined by the claims.

What is claimed is:

1. A method for detecting the content of acetaldehyde in wine samples using fluorescent probes, comprising the following steps:
(1) dispersing fluorescent probes with the structure shown in Formula (I) in organic solvents to obtain a fluorescent probe solution, then, mixing the fluorescent probe solution, a hydrochloric acid solution and a series of acetaldehyde standard solutions with known concentrations respectively for reaction at 0-10° C., and obtaining a mixed system after the reaction; 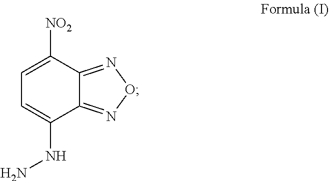 [SMILES: NNc1ccc([N+](=O)[O-])c2nonc12]
(2) measuring the fluorescence intensity of the mixed system on a fluorescence spectrometer, and linearly correlating the fluorescence intensity with the concentration of the corresponding acetaldehyde standard solution to obtain a quantitative detection model; and
(3) mixing the fluorescent probe solution, the hydrochloric acid solution and pretreated wine samples for reaction at 0-10° C. similar to the process in step (1), then, measuring the fluorescence intensity thereof, and calculating the concentration of acetaldehyde in the wine samples by the quantitative detection model obtained in step (2).

2. The method according to claim 1, wherein the volume ratio of the fluorescent probe solution to the hydrochloric acid solution to the acetaldehyde standard solution is 2:1:1.

3. The method according to claim 1, wherein the organic solvents in step (1) are acetonitrile and dimethyl sulfoxide (DMSO), and the volume ratio of the acetonitrile to the DMSO is 10:1.

4. The method according to claim 1, wherein the concentration of the fluorescent probe solution is 600 mg/L.

5. The method according to claim 1, wherein the hydrochloric acid solution is prepared by taking acetonitrile as a solvent.

6. The method according to claim 1, wherein the pH of the hydrochloric acid solution is 2.

7. The method according to claim 1, wherein the concentration range of a series of acetaldehyde standard solutions with known concentrations is 0-200 mg/L.

8. The method according to claim 1, wherein the fluorescence intensity is the fluorescence intensity at the emission wavelength of 553 nm.

9. The method according to claim 1, wherein the quantitative detection model is F_{553 nm}=346.14C+45.17, R²=0.9954, and the unit of C is mg/L.

10. The method according to claim 1, comprising pretreating the wine samples as follows:
adding a drop of defoamer to aerated beer samples or diluting Baijiu, Huangjiu and grape wine samples 25 times with distilled water; then, taking 50 mL, and distilling with a diacetyl distilling apparatus; and stopping receiving when the content of a distillate is close to 10 mL, and complementing to 10 mL with distilled water to obtain a distillate A, that is, the pre-treated wine sample.
